(19) United States
(12) Patent Application Publication
(10) Pub. No.: US 2003/0092979 A1
(43) Pub. Date: May 15, 2003

(54) Non-contact tonometer having fluid pump driven by proportional solenoid

(75) Inventors: David A. Luce, Clarence Center, NY (US); David G. Kelkenberg, Akron, NY (US)

Correspondence Address: George L. Snyder, Jr., Hodgson Russ LLP, One M&T Plaza, Suite 2000, Buffalo, NY, 14203-2391, US

(73) Assignee: Leica Microsystems Inc., Depew, NY (US)

(21) Appl. No.: 10/037,327

(22) Filed: Nov. 9, 2001

(51) Int. Cl. A61B 3/16

(52) U.S. Cl. 600/398

(57) ABSTRACT

A non-contact tonometer comprises a fluid pump system having a linear proportional solenoid for driving a piston of the fluid pump system to generate a fluid pulse in a predetermined and controlled manner. The energizing current to the linear proportional solenoid is dictated by data stored in a digital look-up table.

### BACKGROUND OF THE INVENTION

I. Field of the Invention

The present invention relates generally to ophthalmic instruments, and more particularly to a non-contact tonometer having an improved fluid pump system for generating a fluid pulse.

II. Description of the Related Art

Non-contact tonometers are well-known in the field of ophthalmology for measuring intraocular pressure (IOP) by directing a fluid pulse at the cornea C to cause observable deformation of the cornea. In prior art non-contact tonometers, such as tonometer 10 shown schematically in FIG. 1, the fluid pulse is generated by a piston 12 slidably received by a cylinder housing 14 and axially driven relative to the cylinder housing to compress fluid within a compression chamber 16 defined by the cylinder housing. A plenum chamber 18 directly adjoins compression chamber 14, and a fluid discharge tube 20 is arranged in flow communication with the compression chamber by way of the plenum chamber for directing a fluid pulse along a test axis TA toward cornea C. Measurement of IOP is based on correlation to the pressure within plenum chamber 18 at the moment a predetermined area of the cornea is flattened, a condition known as “applanation”. In order to provide a signal indicative of the occurrence of applanation, a photosensitive detector 30 is positioned in a symmetrically oblique arrangement about test axis TA to receive corneally reflected light from emitter 32, whereby a peak signal is produced by detector 30 when the corneal surface is flat for coherent reflection.

For many years, non-contact tonometers relied exclusively on a rotary solenoid 22 connected via an arm linkage 23 to the piston 12 for driving the piston in its compression stroke. The rotary solenoid 22 was energized by a constant current source 24 under the control of a microprocessor 26, an arrangement that was preferred because it produced a linear increase in plenum pressure as a function of time. This behavior was desirable because the plenum pressure at applanation could be indirectly ascertained in an easy fashion by observing the length of time necessary to achieve applanation. With the use of a rotary solenoid, the force acting on the piston is low at the beginning of the compression stroke and increases during the compression stroke, causing an unwanted delay in the fluid pump.

As pressure sensor technology progressed, the use of a miniature pressure sensor 28 in the plenum chamber 18 to directly monitor plenum pressure replaced the use of an indirect time-based pressure calculation. The analog signals from applanation detector 30 and pressure sensor 28 are digitized by analog-to-digital converter circuits 29 and input to the microprocessor 26 for calculating IOP in a manner well-known to those skilled in the art.

Despite the fact that time-based pressure measurement has been obsolete for nearly a decade, manufacturers of non-contact tonometers have clung to the rotary solenoid as a means for driving the piston. It is also known, however, to use a linear motor for driving the piston in a non-contact tonometer. For example, use of a linear d.c. motor is taught in U.S. Pat. No. 5,048,526 issued Sep. 17, 2001 and U.S. Pat. No. 5,779,633 issued Jul. 14, 1998. Linear motors provide reversible control of the piston movement direction by way of changing the direction of the energizing current in the motor coils.

U.S. Pat. No. 6,159,148 to Luce teaches the use of a rotary solenoid or a linear motor in combination with an increasing, as opposed to a constant, current source. The goal of the invention described in the '148 patent is to provide a non-linear pressure increase in the plenum chamber to reduce the impulse energy associated with the pulse that is responsible for patient discomfort. While this patent teaches the desirability of a non-linear pressure curve, its approach in attaining this goal is limited by difficulty in suitably controlling the output of a rotary solenoid or a standard linear d.c. motor.

### BRIEF SUMMARY OF THE INVENTION

Therefore, it is an object of the present invention to provide a fluid pump system for a non-contact tonometer that is controllable with respect to the force applied to a piston thereof throughout the compression stroke of the piston.

It is another object of the present invention to provide a fluid pump system for a non-contact tonometer that is fast to respond to energizing current.

In view of these and other objects, a non-contact tonometer of the type having a cylinder defining a compression chamber, a piston movable in a forward direction along a stroke axis relative to the cylinder for compressing fluid within the compression chamber, drive means operatively connected to the piston for forcing the piston in the forward direction, energizing means for supplying current to the drive means, and a fluid discharge tube in flow communication with the compression chamber for directing a fluid pulse along a test axis, is improved by providing a linear proportional solenoid in place of a rotary solenoid or standard linear motor as the drive means for the piston. The linear proportional solenoid has an output driving force that is proportional to its energizing current, thereby allowing for desired motion control throughout the compression stroke. In a preferred embodiment, the energizing current is controlled in accordance with a predefined look-up table stored in programmable memory.

### BRIEF DESCRIPTION OF THE SEVERAL VIEWS OF THE DRAWINGS

The nature and mode of operation of the present invention will now be more fully described in the following detailed description of the invention taken with the accompanying drawing figures, in which:

FIG. 1 is a schematic depiction of a non-contact tonometer formed in accordance with known prior art;

FIG. 2 is a schematic depiction of a non-contact tonometer formed in accordance with the present invention;

FIG. 3 is a graph of drive current versus time for the linear proportional solenoid of the present invention; and

FIG. 4 is a schematic diagram of the circuit producing the drive current shown in FIG. 3.

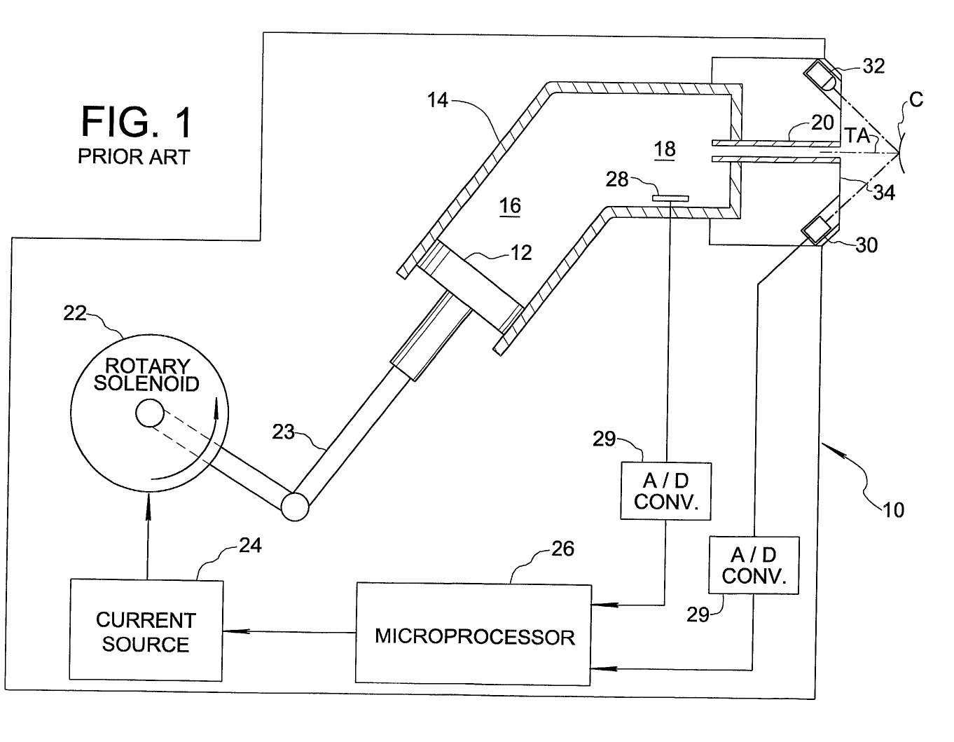

### DETAILED DESCRIPTION OF THE INVENTION

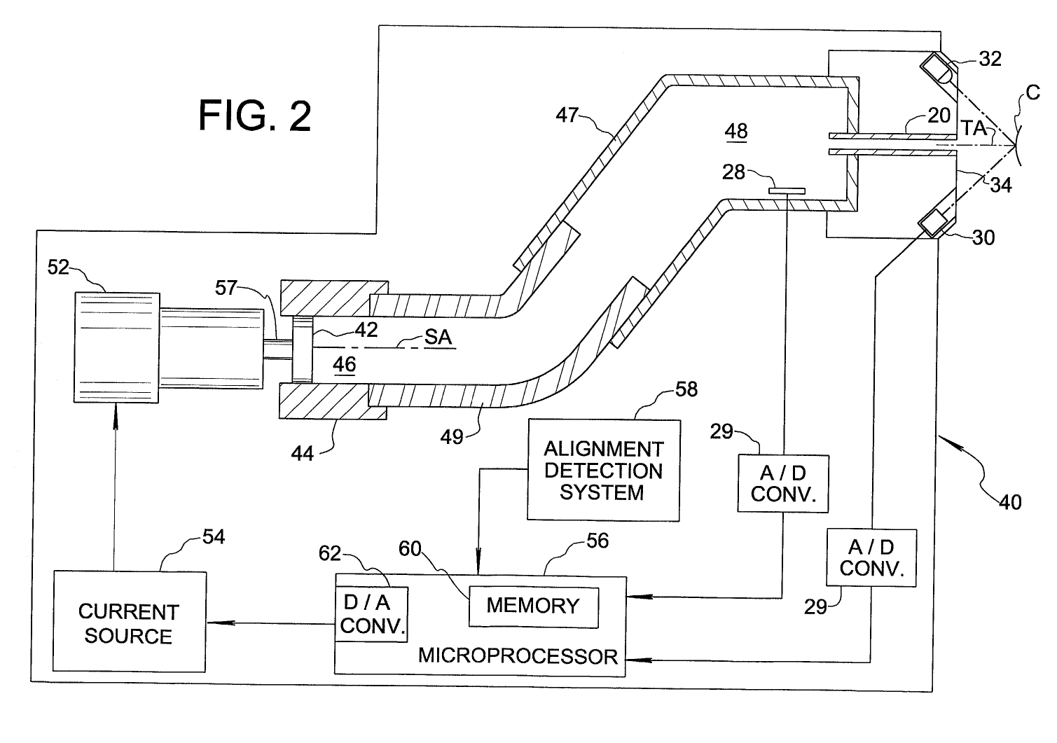

Referring to FIG. 2 of the drawings, a tonometer 40 includes a fluid pump system for generating a fluid pulse used to applanate a patient's cornea during testing. In accordance with a preferred embodiment of the present invention, the fluid pump system comprises a piston 42 axially movable relative to a cylinder 44 along a stroke axis SA for compressing fluid within an internal compression chamber 46 defined thereby, a housing 47 defining an internal plenum chamber 48, a flow tube 49 providing a fluid conduit from compression chamber 46 to plenum chamber 48, and a fluid discharge tube 20 mounted through the wall of housing 47 for guiding pressurized fluid from plenum chamber 48 along test axis TA directed at patient cornea C.

In accordance with the present invention, a linear proportional solenoid 52 is operatively connected to piston 42 for causing axially directed movement of piston 42 relative to cylinder 44. A linear proportional solenoid is a specialized type of linear motor wherein the output driving force is proportional to the energizing current, and is most often used in connection with control valves. Linear proportional solenoid 52 is connected to a current source 54 which supplies energizing current to the linear proportional solenoid under the control of a microprocessor 56. A suitable linear proportional solenoid is a LEDEX® Linear Shift Solenoid Part No. 197887-001. As can be seen in FIG. 2, piston 42 is fixed for travel with a plunger 57 of linear proportional solenoid 52, as by threaded attachment or by fitted attachment with or without mechanical fasteners or adhesives.

Linear proportional solenoid 52 remains de-energized and piston 42 remains at rest until proper positioning of discharge tube 20 relative to cornea C is achieved as determined by an alignment detection system 58 connected to microprocessor 56. Alignment detection system 58 can be any suitable system, for example an alignment system taught in commonly owned U.S. Pat. No. 4,881,807 issued Nov. 21, 1989, the disclosure of which is hereby incorporated by reference. Once alignment is achieved, microprocessor 56 provides a signal used by current source 54 to provide the driving current according to a preprogrammed ramp form, as will be described below.

The use of linear proportional solenoid 52 enables programmable control of the force driving piston 42 through the compression stroke. More specifically, a lookup table stored in a programmable memory 60 associated with microprocessor 56 includes digital information describing the desired current versus time relationship, which information can be used to actually generate the energizing current ramp.

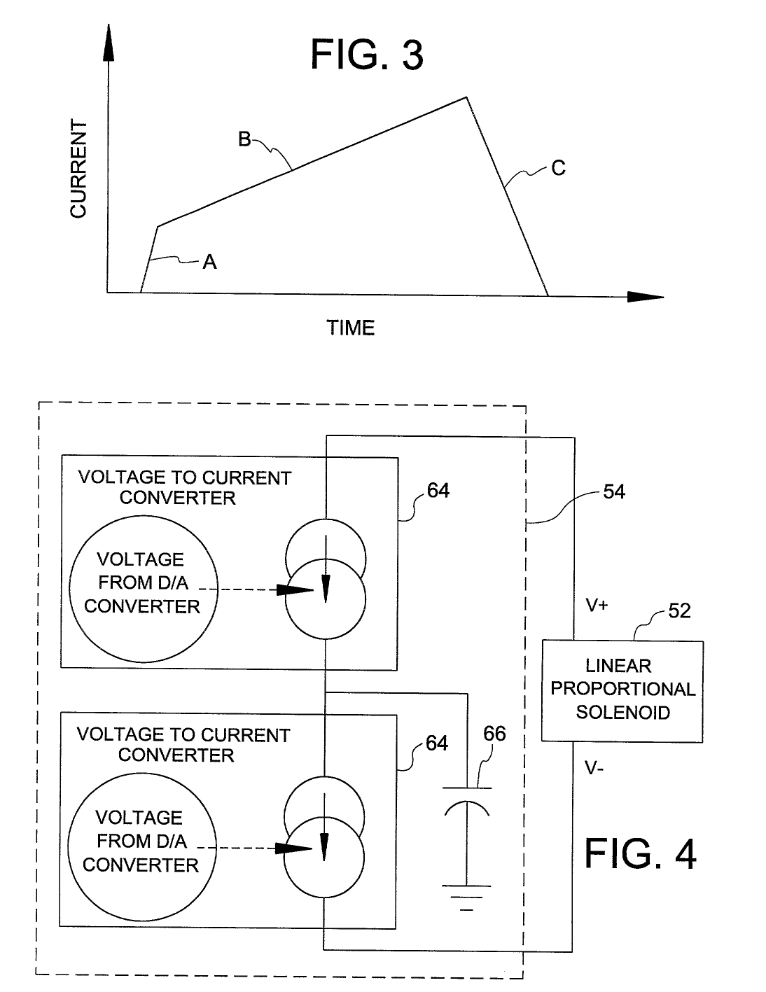

By way of non-limiting example, FIG. 3 shows a presently favored relationship of drive current versus time as generated by current source 54 for the compression stroke of piston 42. The shape includes three stages A, B, and C each defined by a straight line segment. Stage A is steeply sloped to quickly accelerate the piston from its resting position, thereby taking advantage of the fact that linear proportional solenoid has a fast response and almost no starting delay. Stage B is more moderately sloped to smoothly increase the driving force on piston 42, whereby a nonlinear pressure increase is realized in plenum chamber 48. Stage C is a steeply sloped discharge stage which decreases the electromotive force applied to piston 42 back to zero, whereby the piston returns with solenoid plunger 57 back to a reference position under urging of a spring (not shown) located within the housing of linear proportional solenoid 52.

The waveform shown in FIG. 3 is achieved using a lookup table having only nine data points. These data points correspond to the slope, the start time, and the stop time of each of the three line segments corresponding to stages A, B, and C. The data is converted to analog voltage signal form by a digital-to-analog converter 62 either onboard or separate from microprocessor 56, and the voltage signal is applied to current source 54. FIG. 4 provides a conceptual schematic diagram of current source 54, which operates by controlling the charge and discharge current in a capacitor 66. Current source 54 comprises a pair of voltage-to-current converter circuits 64, one for charge and the other for discharge.

As will be appreciated from the foregoing description, the look-up table values stored in programmable memory 60 can be adjusted to provide customized control of the piston stroke. For example, the current can be held constant for a period of time at the end of the stroke at a current level that achieves equilibrium between the electromotive drive force and the return spring force, such that the plunger 57 and piston 42 are held in place. Then the current can be slowly decreased to allow piston 42 and plunger 57 to return in a controlled manner to the starting reference position. This manner of control helps minimize undesirable drawback of eye fluids into the discharge tube 20.

What is claimed is:

1. In a non-contact tonometer having a cylinder defining a compression chamber, a piston movable in a forward direction along a stroke axis relative to said cylinder for compressing fluid within said compression chamber, drive means operatively connected to said piston for forcing said piston in said forward direction, energizing means for supplying current to said drive means, and a fluid discharge tube in flow communication with said compression chamber for directing a fluid pulse along a test axis, the improvement comprising:
said drive means comprising a linear proportional solenoid.

2. The improvement according to claim 1, wherein said energizing means supplies said current according to a programmable look-up table.

3. The improvement according to claim 1, wherein said programmable look-up table includes data as to the slope, start time, and stop time of one or more linear segments defining a current versus time relationship.
